(19) United States
(12) Patent Application Publication
(10) Pub. No.: US 2013/0303945 A1
(43) Pub. Date: Nov. 14, 2013

(54) ELECTROMAGNETIC TIP SENSOR

(71) Applicant: INTUITIVE SURGICAL OPERATIONS, INC., Sunnyvale, CA (US)

(72) Inventors: Stephen J. Blumenkranz, Los Altos Hills, CA (US); Dorin Panescu, San Jose, CA (US)

(73) Assignee: Intuitive Surgical Operations, Inc., Sunnyvale, CA (US)

(21) Appl. No.: 13/893,460

(22) Filed: May 14, 2013

Related U.S. Application Data
(60) Provisional application No. 61/646,608, filed on May 14, 2012.

(51) Int. Cl. A61M 25/00

(52) U.S. Cl. CPC A61M 25/0067
USPC 600/585

(57) ABSTRACT

A medical instrument having a distal tip through which a lumen extends can employ an electromagnetic sensor including a coil that is in the distal tip and winds around the lumen or a coil that is in the distal tip and defines an area having a normal direction that is perpendicular to an instrument axis that extends along the lumen. Three coils can be oriented so that normal directions of the areas defined by the coils are along three orthogonal axes.

### CROSS-REFERENCE TO RELATED APPLICATIONS

This patent document claims benefit of the earlier filing date of U.S. provisional patent application 61/646,608, filed May 14, 2012, which is hereby incorporated by reference in its entirety.

### BACKGROUND

Electromagnetic sensors (EM) sensors can be used to measure the position and orientation of the structure on which the EM sensor is mounted and can be made compact enough for use in minimally invasive medical instruments. Existing EM sensors typically include two or more coils of electrical wire with coil axes oriented at an angle (usually less than) 90° to each other. The coils act as antennae. In use, a field generator generates an electromagnetic field that induces electrical signals in the coils of the EM sensors, and the electrical signals can be monitored and analyzed to deduce the position and orientation of the EM sensor with respect to the field generators. Multiple degrees of freedom of position and orientation of a portion of the medical instrument within a patient can thus be measured.

EM sensors typically employ long thin coils when used in minimally invasive medical devices. The coils may be thin enough to allow positioning of the coils within the walls of a long, thin medical instrument, but the small diameter of the coils may make measurements subject to noise and error, particularly when the EM sensors are near ferrous metal structures that may be moving within a medical instrument. Further, measurement of some degrees of freedom, e.g., a roll angle, with an EM sensor requires at least two coils at a non-zero angle, but the requirement of a compact sensor package generally requires a non-orthogonal orientation of the coils and may limit accuracy. Even when the angle between the coils is less than 90°, making an EM sensor small enough to fit in the distal tip of some medical instruments can be difficult. For example, a lung catheter may require a distal tip that is smaller than about 3 mm in diameter to fit within a small bronchial tube, and that distal tip needs to include a lumen with an opening as large as possible in order to accommodate a lung biopsy tool. The EM sensor thus needs to compete for space with the main lumen of the catheter, and even an EM sensor with a diameter of 1 mm may be too large to fit within the distal tip of an instrument. However, if an EM sensor is positioned away from the distal tip, extrapolation or relative measurements from the location of the EM sensor to the location of the distal tip can increase the error in the measurement of the position and orientation of the distal tip.

### SUMMARY

In accordance with an aspect of the invention, a medical instrument such as a catheter having a distal tip through which a lumen extends can employ an electromagnetic sensor including a coil that is in the distal tip and winds around the lumen or a coil that is in the distal tip and defines an area having a normal direction that is perpendicular to an axis that extends along the lumen of the instrument. Three such coils can be oriented so that normal directions of the areas defined by the coils are along three orthogonal axes.

One specific embodiment of the invention is a medical instrument having a main tube with a distal tip through which a lumen of the main tube extends. An electromagnetic sensor for the medical instrument includes a coil that is in the distal tip and defines an area through which the lumen passes.

Another specific embodiment is a medical instrument including a main tube having a distal tip. An electromagnetic sensor for this embodiment includes a coil that is in the distal tip and defines an area positioned such that a radial axis that extends from a central axis of the main tube passes through the area.

Yet another embodiment is a medical instrument including a main tube and an electromagnetic sensor. The electromagnetic sensor includes a first coil and a second coil in a distal tip of the main tube. The first coil defines a first area having a first normal direction, and the second coil defines a second area having a second normal direction that is perpendicular to the first normal direction.

Still another embodiment of the invention is a method that includes placing an instrument in a patient and generating a variable magnetic field with a known orientation with respect to anatomy of the patient. The instrument defines an interior lumen and has a distal tip containing a coil of an electromagnetic sensor. The coil may wind around the interior lumen or may define an area positioned such that a radial axis that extends from a central axis of the lumen passes through the area. In either case, an electrical signal induced in the coil can be used to measure and compute a position or orientation of the distal tip.

### BRIEF DESCRIPTION OF THE DRAWINGS

FIG. 1 shows a minimally invasive medical instrument having an electromagnetic sensor in its distal tip.

FIG. 2 shows an embodiment of a steerable segment that can be employed in the system of FIG. 1.

FIGS. 3A and 3B respectively show transparent perspective and axial views of the distal tip of a catheter including orthogonal EM antenna coils surrounding a central tool lumen.

FIG. 4 is an axial view of a distal tip of a medical instrument having a center lumen, a thin axial-facing EM antenna, and radial-facing EM antenna coils.

FIG. 5 is an axial view of a distal tip of an instrument having an axial-facing EM antenna surrounding a central lumen and radial-facing EM antennae that are not orthogonal to each other.

FIG. 6 is an axial view of a distal tip of an instrument having an axial-facing EM antenna defining an area within a wall of the instrument and radial-facing EM antennae that are not orthogonal to each other.

FIGS. 7A, 7B, and 7C are axial views of the distal tips of medical instruments with using alternative two-coil configurations for electromagnetic sensing of six degrees of freedom of the respective distal tips.

FIG. 8 shows a transparent side view of a distal tip of a medical instrument employing sensing coils that surround a central lumen of the instrument and define flux areas with normal directions at non-zero angles with a central axis of the instrument.

FIG. 9 is an axial view of the distal tip of a probe that may be deployed through a catheter.

Use of the same reference symbols in different figures indicates similar or identical items.

### DETAILED DESCRIPTION

An EM sensor at the tip of a medical instrument can include a coil defining an area through which a central axis of the instrument passes and one or more coils defining areas through which radial axes extending through the central axis pass. For example, an EM sensor at the tip of a catheter can include a coil of wire that wraps around a main lumen of the catheter. The coil may be oriented so that a normal direction of the area defined by the coil is parallel to the central axis of the main lumen or at a non-zero angle to the central axis. Two coils defining areas through which radial axes pass may have normal directions perpendicular to the central axis of the main lumen and can also be positioned so that the normal directions of the areas of the two coils are perpendicular to each other. Accordingly, an EM sensor at the tip of the medical instrument can include three coils associated with three orthogonal axes. The orthogonal coils in the tip may provide an ideal set of induced signals for precision determination of the position and orientation of the tip. Further, the orientation of the coils may allow for greater coil diameter and create a coil or antenna that produces a larger magnitude electrical signal and improves the signal-to-noise ratio of the electrical signal. Improvements in the signal-to-noise ratio may permit shorter sample integration time for position and orientation measurements, resulting in a higher sample rate and leading to improved servo performance for closed loop control of the distal tip. The improved signal-to-noise ratio may also enable more accurate navigation of a biopsy catheter to suspected tumors identified in CT or MRI images, which could result in higher yield of biopsy tissue specimens from suspect tumor bodies. The tip mounted EM sensor, which may be used for a lung catheter, may also be used with similar advantages in catheters and other medical devices for diagnosis or treatment in cardiology, peripheral vascular disease, neurology, or other disease areas.

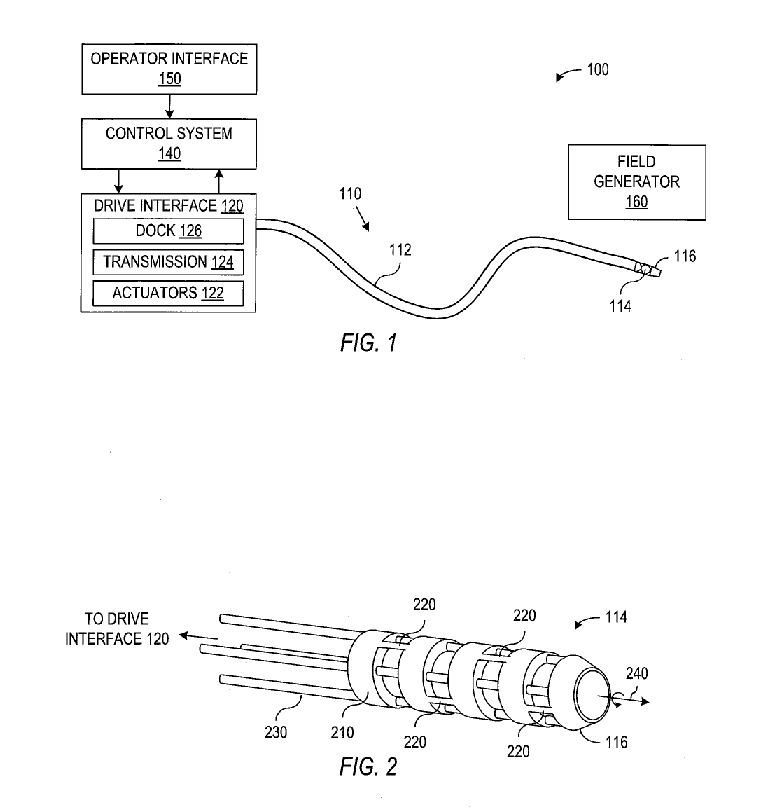

FIG. 1 schematically illustrates a medical system 100 in accordance with one embodiment of the invention. In the illustrated embodiment, medical system 100 includes a flexible device 110, a drive interface 120, control system 140, an operator interface 150, and a field generator 160 for a sensing system.

Device 110, in the illustrated embodiment, may be a flexible device such as a lung catheter that includes a flexible main shaft 112 with one or more lumens. For example, main shaft 112 may include a main lumen sized to accommodate interchangeable probes (not shown). Such probes can include a variety of a camera or vision systems or biopsy tools that may be deployed through or removed from device 110. Additionally, main shaft 112 may incorporate a steerable distal section 114 that is similarly operable using actuating tendons that attach to steerable section 114 and run from steerable section 114 at the distal end of main shaft 112, through main shaft 112, to the proximal end of main shaft 112.

Main shaft 112 can be implemented using flexible structures such as braid reinforced tubing including a woven wire tube with inner or outer layers of a flexible or low-friction material such as polytetrafluoroethylene (PTFE). An exemplary embodiment of device 110 is a lung catheter, where device 110 would typically be about 60 to 80 cm long or longer. During a medical procedure such as a lung biopsy, at least a portion of main shaft 112 and all of steerable section 114 may be inserted along a natural lumen such as an airway of a patient, and drive interface 120 may operate steerable section 114 by pulling on actuating tendons, e.g., to steer device 110 during insertion. After insertion, drive interface 120 may pull the tendons to position and orient steerable section 114 and particularly a distal tip 116 of steerable section 114 in a pose required for a medical procedure. Distal tip 116 contains sensor coils as described further below, and a control system 140 may employ measurements of the position and orientation of distal tip 116 during control or use of device 110.

Steerable section 114 is remotely controllable and particularly has a pitch and a yaw motion direction that can be controlled using actuating tendons, e.g., pull wires or cables, and may be implemented as a tube of flexible material such as Pebax. In general, steerable section 114 may be more flexible than the remainder of main tube 112, which assists in isolating actuation or bending to steerable section 114 when drive interface 120 pulls on the actuating tendons. Device 110 can also employ additional features or structures such as use of Bowden cables for actuating tendons to prevent actuation from bending the more proximal portion of main tube 112. In general, the actuating tendons are attached to different points around the perimeter of steerable section 114. For example, FIG. 2 shows one specific embodiment in which steerable section 114 is made from a tube 210 that is cut to create flexures 220. Tube 210 in the illustrated embodiment may define a main lumen for probe systems and smaller lumens for actuating tendons 230. In the illustrated embodiment, four actuating tendons 230 attach to a base of distal tip 116 at locations are that 90° apart around a central axis 240 of steerable section 114. In operation, pulling harder on any one of tendons 230 tends to cause steerable section 114 to bend in the direction of that tendon 230.

Drive interfaces 120 of FIG. 1, which pulls on actuating tendons 230 to operate steerable section 114, includes a mechanical system or transmission 124 that converts the movement of actuators 122, e.g., electric motors, into movements of (or tensions in) actuating tendons 230. The movement and pose of steerable section 114 can thus be controlled through selection of drive signals for actuators 122 in drive interface 120. In addition to manipulating the actuating tendons, drive interface 120 may also be able to control other movement of device 110 such as a range of motion in an insertion direction and rotation or roll of the proximal end of device 110, which may also be powered through actuators 122 and transmission 124. Backend mechanisms or transmissions that are known for flexible-shaft instruments could in general be used or modified for drive interface 120. Drive interface 120 may further include a dock 126 that provides a mechanical coupling between drive interface 120 and device 110 and links the actuating tendons 230 to transmission 124. Dock 126 may additionally contain an electronic interface for receiving, converting, and/or relaying sensor signals received from EM sensors in distal tip 116.

Control system 140 controls actuators 122 in drive interface 120 to selectively pull on the actuating tendons 230 as needed to actuate or steer steerable section 114. In general, control system 140 operates in response to commands from a user, e.g., a surgeon or other medical personnel using operator interface 150, and in response to measurement signals such as from EM sensors in distal tip 116. Control system 140 may in particular include or execute sensor logic that analyzes signals (or digitized versions of signals) from the EM sensors in distal tip 116 and determines or measures the position and orientation of distal tip 116. Control system 140 may be implemented using a general purpose computer with suitable software, firmware, and/or interface hardware to interpret signals from operator interface 150 and EM sensors and to generate control signals for drive interface 120.

Operator interface 150 may include standard input/output hardware such as a display, a keyboard, a mouse, a joystick, or other pointing device or similar I/O hardware that may be customized or optimized for a surgical environment. In general, operator interface 150 provides information to the user and receives instructions from the user. For example, operator interface 150 may indicate the status of system 100 and provide the user with data including images and measurements made by system 100. One type of instruction that the user may provide through operator interface 150, e.g., using a joystick or similar controller, indicates the desired movement or position and orientation of steerable section 114, and using such input and sensor feedback from distal tip 116, control system 140 can generate control signals for actuators in drive interface 120.

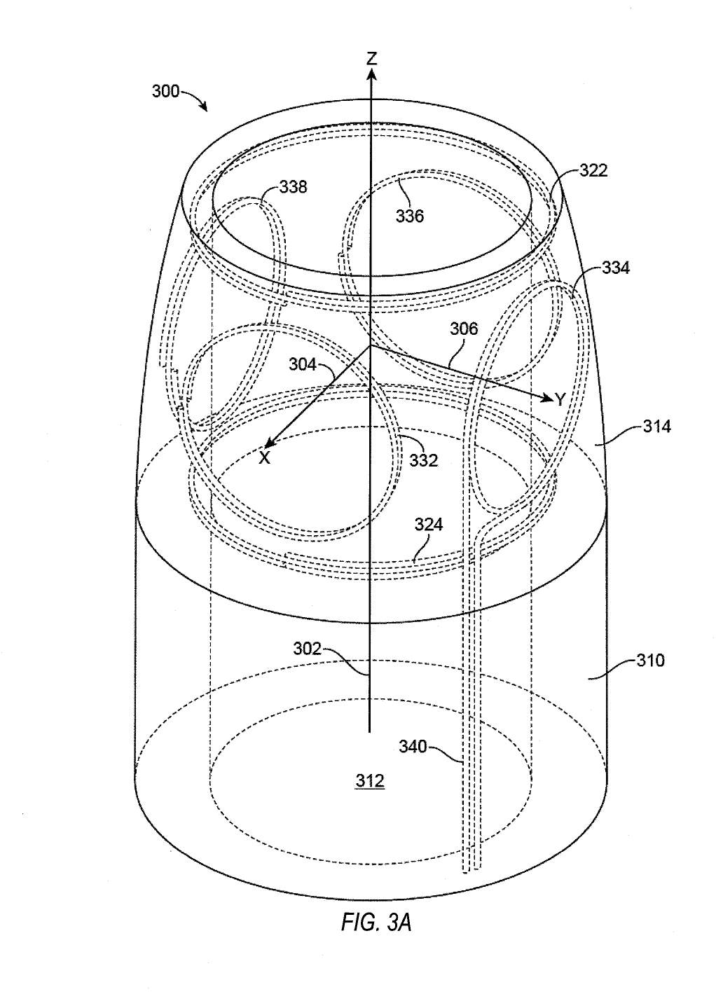

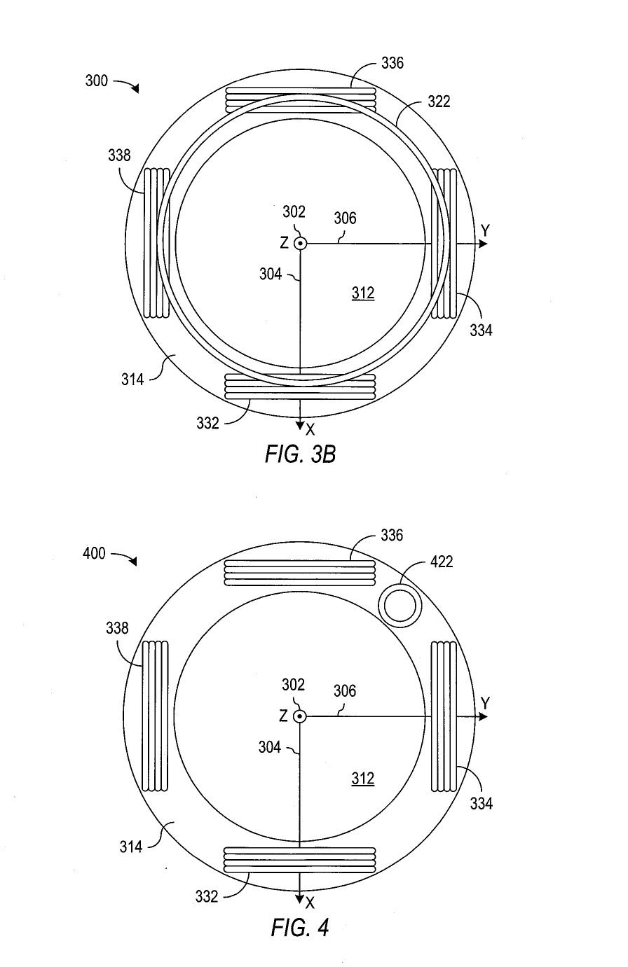

Field generator 160 and one or more EM sensors in distal tip 116 can be used to measure a pose of distal tip 116. FIGS. 3A and 3B respectively show transparent perspective and axial views of an embodiment of a distal tip 300, which illustrates one configuration for sensing coils in distal tip 116 of FIG. 1. As shown in FIG. 3A, distal tip 300 is at the end of a guide structure 310 through which a tool channel lumen 312 passes. Guide structure 310 may, for example, be similar or identical to main tube 112 or steerable section 114 of FIG. 1. Six coils 322, 324, 332, 334, 336, and 338 are around tool channel lumen 312 and are encapsulated in a wall 314 of tip 300. In particular, wall 314 may be made of a non-ferromagnetic material such as pebax, urethane, polyamide or other polymeric material in which EM sensor antenna coils 322, 324, 332, 334, 336, and 338 are embedded. Coils 332, 334, 336, and 338 could optionally contain a ferromagnetic core, e.g., an iron disk within the area defined by the wire loops of coils 332, 334, 336, and 338. Coils 322, 324, 332, 334, 336, and 338 being in distal tip 300 are in position to directly measure the position and orientation of distal tip 300. In contrast, some prior systems may position EM sensors more proximally in an instrument where more space may be available and then extrapolate or use relative measurements to determine the pose of the distal tips. Such techniques may be subject to propagation of errors.

Medical instruments often need a measurement of the pose of the extreme distal tip of the instrument because that pose may control steering of the instrument and because the extreme distal tip is generally where the instrument must precisely interact with tissue. Coils 322, 324, 332, 334, 336, and 338 are in distal tip 300 at the distal end of a medical instrument to provide particularly useful and accurate measurements of the pose of distal tip 300. More generally, coils 322, 324, 332, 334, 336, and 338 may be positioned so that any extrapolation from the position and orientation directly measured to an extreme end of the instrument is along a well defined length and the measured orientation. In this sense, the distal tip may, for example, include the most distal discrete controllable part, e.g. a rigid link, of a medical instrument rather than only the distal portion of the instrument within some distance, e.g., less than 2 or 3 mm, from the extreme distal end of the instrument.

Coils 322, 324, 332, 334, 336, and 338 are encapsulated in tip 300 for EM sensing. In particular, EM sensor antenna coils 322, 324, 332, 334, 336, and 338 can be advantageously distanced from ferromagnetic metal structures that move relative to distal tip 300 and may cause noise in the induced signals. Increased signal to noise ratio beneficially comes from the larger diameter and internal area of the coils because the received signal is correspondingly larger than stray effects that can be induced in the lead wires and can arise in the signal processing circuitry. In addition, placement of the coils being in a discrete rigid distal tip can reduce or eliminate extrapolation error in estimating the extreme tip position and orientation from position and orientation measurements made at a defined distance back from the extreme distal tip.

Coils 322 and 324 are oriented so that the areas defined by loops of wire in coils 322 and 324 may have a normal direction along central axis 302, but alternatively the normal direction to areas defined by coils 322 and 324 may be a non-zero angle to central axis 302. Further, coils 322 and 324 may include wire that is wound around tool channel lumen 312 so that central axis 302 and tool channel lumen 312 passes through coils 322 and 324. The diameters of coils 322 and 324 may thus be larger than the diameter of tool channel lumen 312 and may be almost as large as the diameter of distal tip 300. In contrast, the diameter of a coil that is similarly parallel to central axis 302 but offset from central axis 302 and sealed within the wall of distal tip 300 may be no larger than the thickness of the wall. Since the area of a coil increases in proportion to the square of the diameter of the coil, coil 322 can provide a much greater area and corresponding larger magnitude sensing signal induced by variation of a larger amount of magnetic flux through the coil 322.

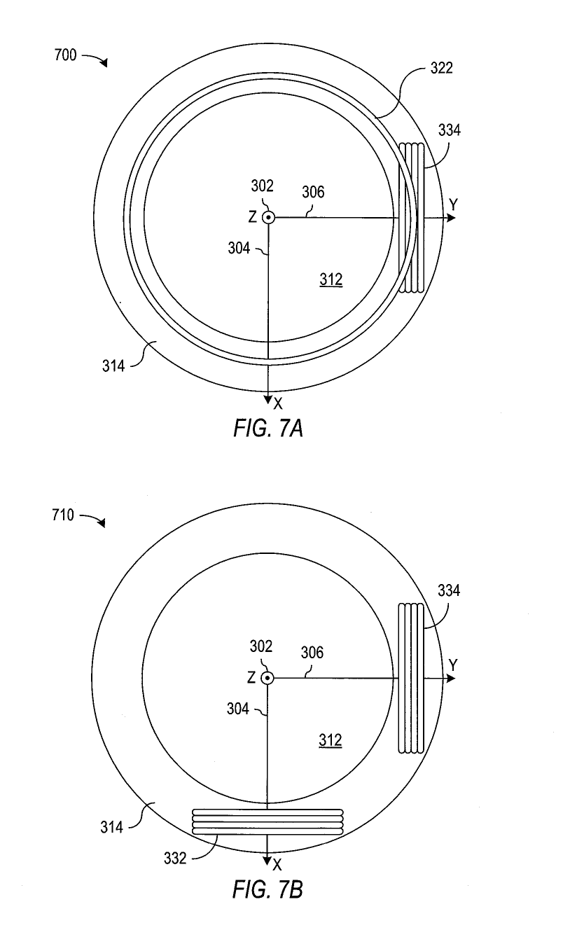

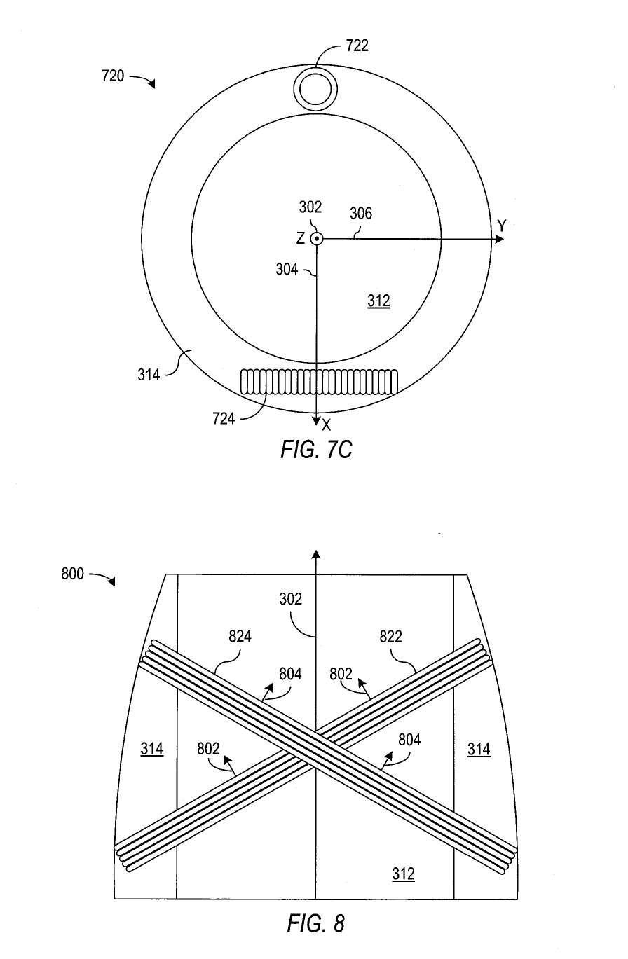

Coils 322 and 324 when centered on central axis 302 may provide further advantages when compared to smaller diameter coils (e.g., coils 722 and 724 of FIG. 7) that are in the walls of a catheter. In particular, a single coil such as coil 322 can be used to measure a position and a pointing direction, e.g., pitch and yaw angles of distal tip 300, but a cylindrically symmetrical coil is unable to distinguish roll angles about the symmetry axis of the coil. Accordingly, if coil 322 is a cylindrically symmetric helical coil centered on central axis 302, signals from coil 322 can provide information about five degrees of freedom, not including the roll angle about central axis 302, and the five degrees of freedom measured using coil 322 are simply related to the degrees of freedom of distal tip 300 relative to central axis 302. In contrast, a thin coil defining an area within wall 314 generally needs to be offset from axis 302 by about the radius of tip 300, which provides a measurement for a location that is offset from the central axis. Thus, the five degrees of freedom that a thin coil measures may depend on mixtures of the orientation and position of distal tip 300.

Coils 332, 334, 336, and 338 have areas with normal directions that are directed outward (or inward) from central axis 302 or tool channel lumen 312 so that a radial axis 304 or 306 passes through coils 332, 334, 336, and 338. Although coils 332, 334, 336, and 338 are illustrated in FIGS. 3A and 3B, as flat or cylindrically symmetric coils, coils 332, 334, 336, and 338 may be saddle-shaped to allow for a greater coil area within wall 314. The areas defined by loops of wire in coils 332, 334, 336, and 338 may have average normal directions that are perpendicular to central axis 302. Accordingly, the normal directions of the areas defined by coils 332, 334, 336, and 338 can be perpendicular or orthogonal to the normal directions for the areas defined by coils 322 and 324. Coils 332 and 336 or 334 and 338 can be centered on axis 304 or 306 that extend from central axis 302. Further, radial axis 304 and coils 332 and 336 can be oriented perpendicular to radial axis 306 and coils 334 and 338. Coils 322, 324, 332, 334, 336, and 338 can thus provide an EM sensor with sensing coils along three orthogonal axes. More generally, not all of coils 322, 324, 332, 334, 336, and 338 are needed to provide sensing along three orthogonal axes. Three coils, e.g., coils 322, 332, and 334, would be sufficient to provide sensing along three orthogonal axes.

Coils 322, 324, 332, 334, 336, and 338 may have lead wires that extend back through guide structure 310 to an instrument interface or control system such as described above with reference to FIG. 1. FIG. 3A shows lead wires 340 for coil 338. Lead wires 340 may form a twisted pair or employ shielding to reduce noise that may be induced along the lengths of lead wires 340. Although FIG. 3A for ease of illustration shows only one pair of lead wires 340, each coil 322, 324, 332, 334,336, or 338 may have similar lead wires that extend back through guide structure 310. To reduce the number of pairs of lead wires 340, two or more coils may be connected together within distal tip 300 to effectively act as a single coil. For example, coils 322 and 324 may both define areas with normal directions along central axis 302 and may be connected together to act as a single coil generating a single induced electrical signal. Similarly, coils 332 and 336, which may define areas with normal directions along radial axis 304, may be connected together in distal tip 300, and coils 334 and 338, which may define areas with normal directions along radial axis 306, may be connected together in distal tip 300.

Known analysis techniques can use the induced signals generated in the coils shown in FIGS. 3A and 3B to determine the pose of distal tip 300. For example, U.S. Pat. No. 7,197,354, entitled “System for Determining the Position and Orientation of a Catheter”; U.S. Pat. No. 6,833,814, entitled “Intrabody Navigation System for Medical Applications”; and U.S. Pat. No. 6,188,355, entitled “Wireless Six-Degree-of-Freedom Locator” describe the operation of some EM sensor systems and are hereby incorporated by reference in their entirety. U.S. Pat. No. 7,398,116, entitled “Methods, Apparatuses, and Systems useful in Conducting Image Guided Interventions,” U.S. Pat. No. 7,920,909, entitled “Apparatus and Method for Automatic Image Guided Accuracy Verification,” U.S. Pat. No. 7,853,307, entitled “Methods, Apparatuses, and Systems. Useful in Conducting Image Guided Interventions,” and U.S. Pat. No. 7,962,193, entitled “Apparatus and Method for Image Guided Accuracy Verification” further describe systems and methods that can use electromagnetic sensing coils in guiding medical procedures and are also incorporated by reference in their entirety.

A sensing operation employing the EM sensor system of FIGS. 3A and 3B can include operating a field generator to generate an electromagnetic field or a time varying magnetic field having a known orientation to the anatomy of a patient. The field generator may, for example, have a measured position and orientation relative to a patient and include orthogonal groups of parallel wires through which known or measured electrical currents are sequentially sent. The resulting time variation of the magnetic field can induce a current or voltage signal in each sensing coil 322, 324, 332, 334, 336, and 338, where the magnitude of the induced electrical signal depends on the time derivative of the magnetic flux through the coil. Analysis of the induced signals in coils 322 and 324 can provide measurements of five degrees of freedom of distal tip, not including a roll angle about central axis 302. Similarly, analysis of the induced signals in coils 332 and 336 or coils 334 and 338 can provide measurements of five degrees of freedom of distal tip not including a rotation angle about radial axis 304 or 306. The resulting measurements can be combined to determine all six degrees of freedom of distal tip 300, and the orthogonal nature of the normal directions associated with the sensing coils and with the unmeasured rotation angle for each pair of coils may optimize the accuracy of the 6-DoF measurement. More generally, two coils having non-parallel axes may be sufficient for a 6-DoF measurement. For example, coil 322 or 324 and any one of coils 332, 334, 336, and 338 would be sufficient for a 6-Dof measurement, or one of coils 332 and 336 used with one of coils 334 and 338 would be sufficient for a 6-Dof measurement. However, use of more coils with orientations along three orthogonal axes 302, 304, and 306 as shown in FIG. 3A or 3B may provide better accuracy.

FIG. 4 shows an axial view of a distal tip 400 having another configuration for EM sensing coils suitable for use in distal tip 116 of FIG. 1. When compared to the arrangement of coils in distal tip 300 of FIG. 3B, distal tip 400 employs an axial-facing sensing coil 422 defining an area that fits within wall 314 in place of an axial-facing coil 322 that surrounds central axis 302. Axial-facing sensing coil 422 is used with radial-facing coils 332, 334, 336, and 338, which can have an orthogonal configuration as described above with reference to FIGS. 3A and 3B. Coils 422, 332, 334, 336, and 338 thus may provide flux areas with normal directions along three orthogonal axes, which may provide more accurate measurements. More generally, two coils having non-parallel axes may be sufficient for a 6-DoF measurement.

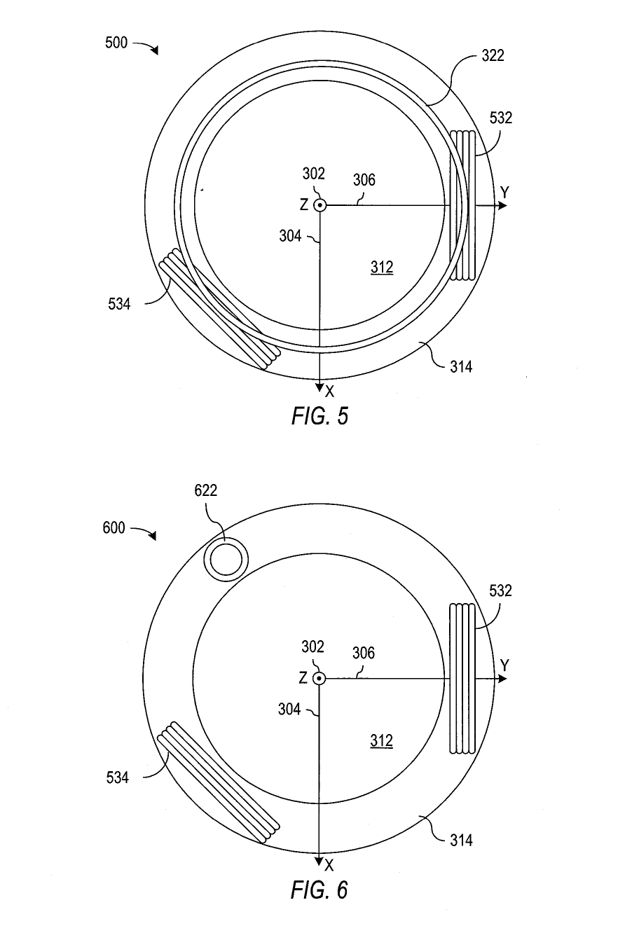

FIG. 5 shows a distal tip 500 of an instrument employing an axial-facing coil 322 through which a tool channel lumen 312 and central axis 302 passes. Distal tip 500 further includes radial-facing coils 532 and 534, which are oriented so that radial axes passing through axis 302 pass through respective areas defined by coils 532 and 534. In the specific configuration of FIG. 5, coils 532 and 534 define areas having respective normal directions that are perpendicular to the normal direction of the area defined by coil 322. However, the normal directions of the areas defined by coils 532 and 534 are not perpendicular to each other. In general, coils that provide perpendicular measurements may provide the most accurate measurements of at least some degrees of freedom. Coils defining areas with normal directions that are non-orthogonal may be employed for the same measurements and may leave space in wall 314 for other structures (not shown).

FIG. 6 shows a distal tip 600 that is substantially identical to distal tip 500, except that axial-facing coil 322 which surrounds tool channel lumen 312 in FIG. 5 is replaced in FIG. 6 with an axial-facing coil 622 defining an area within wall 314 of distal tip 600.

FIGS. 7A, 7B, and 7C respectively illustrate distal tips 700, 710, and 720 of instruments using two-coil EM tip sensors. Two coils that are not parallel are generally sufficient for EM sensing of six degrees of freedom (e.g., position and orientation) of a distal tip. Distal tip 700 of FIG. 7A employs an axial-facing coil 322 and a radial-facing coil 334. Axial-facing coil 322 defines an area containing central axis 302 and tool channel lumen 312, and radial-facing coil 334 defines an area through which a radial axis 306 extending from central axis 302 passes. Distal tip 710 of FIG. 7B does not include an axial-facing coil but instead employs two radial-facing coils 332 and 334. Radial-facing coil 332 defines an area through which a radial axis 304 extending from central axis 302 passes, and radial-facing coil 334 defines an area through which a radial axis 306 extending from central axis 302 passes.

Distal tip 720 of FIG. 7B employs coils 722 and 724 that define areas enclosed with wall 314 of distal tip 720. In the illustrated configuration, coil 722 is an axial-facing coil, i.e., defines an area with a normal direction along axis 302, and coil 724 is oriented perpendicular to central axis 302 and defines an area with a normal direction along axis 306. The dimensions of coils 722 and 724 are mostly limited by the annular area of a cross-section of wall 314. More specifically, the diameters of coils 722 and 724 are limited by the thickness of wall 314, and the length of coil 724 is limited by the chord lengths that fit within in the annular area. The length of coil 722 may be less restricted because coil 722 extends in the direction of the length of the instrument. The available length of coil 724 may be increased by altering the illustrated configuration by rotating coil 724 about axis 304. Coil 722 can be rotated about axis 304 by the same angle as coil 724 to maintain the perpendicular relationship between coils 722 and 724.

The coil configurations of FIGS. 7A, 7B, and 7C are subject to variation. For example, axial-facing coil 322 of FIG. 7A through which central axis 302 and lumen 312 pass can be replaced with a thin axial-facing coil such as coil 722 of FIG. 7C. Similarly, a radial-facing coil such as coil 334 can be replaced with a coil such as 724, which is along a chord of the cross-section of the distal tip. Compared to distal tip 720, distal tips 700 and 710 have the advantage employing coils 322, 332, or 334 in which each loop of wire defines a much greater area than the area of a wire loop in coil 722 or 724.

FIG. 8 shows a distal tip 800 with yet another configuration of EM sensing coils 822 and 824 that are in a wall 314 of distal tip 800 and surround a central axis 302 and a tool channel lumen 312. Coils 822 and 824 define areas with respective normal directions 802 and 804 at non-zero angles with central axis 302. When normal directions 802 and 804 are not parallel, coils 822 and 824 may be sufficient for measurement of six degrees of freedom of distal tip 800. Coils 822 and 824 may provide the most accurate measurement of at least some of the six degrees of freedom when normal directions 802 and 804 are perpendicular. For example, normal direction 802 may be at an angle of +45° with axis 302, and normal direction 804 may be at an angle of −45° with axis 302, so that normal directions 802 and 804 are perpendicular to each other.

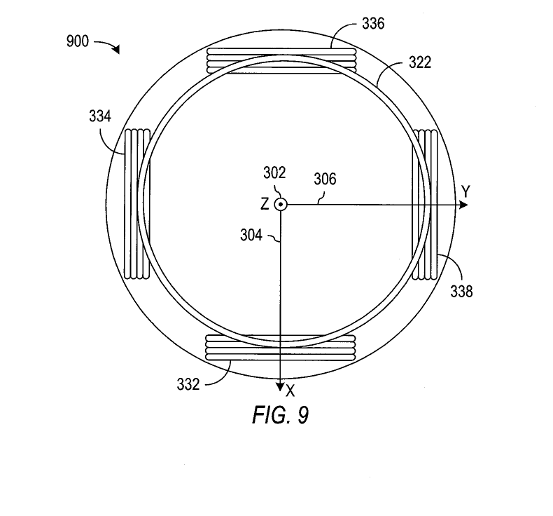

The sensing coil configurations described above are primarily described for the distal tips of medical instruments such as catheters that include central lumens, e.g., a tool channel lumen through which tools or probes can be inserted or removed. However, the EM sensing systems described above can more generally be used in other types of medical instruments or probes. For example, FIG. 9 shows a distal tip 900 including a coil 322 through which a central axis 302 passes and radial-facing coils 332, 334, 336, and 338 through which radial axes 304 and 306 pass. The configuration of coils 322, 332, 334, 336, and 338 may be as described above with reference to FIG. 3B, except that distal tip 900 does not include a tool channel lumen. Instead of a central lumen, distal tip 900 may include a central structure (not shown) that implements a medical function other than guiding a medical tool. Coils 322, 332, 334, 336, and 338 can then be positioned around the central structure as shown, or other configurations of sensing coils such as described above could alternatively be employed in the distal tip of a probe.

Although particular implementations have been disclosed, these implementations are only examples and should not be taken as limitations. Various adaptations and combinations of features of the implementations disclosed are within the scope of the following claims

What is claimed is:

1. A medical instrument comprising:
a main tube having a distal tip through which a lumen of the main tube extends; and
an electromagnetic sensor including a first coil that is in the distal tip and defines a first area through which the lumen passes.

2. The instrument of claim 1, wherein the first area has a first normal direction that is parallel to a central axis of the lumen.

3. The instrument of claim 1, wherein the first area has a first normal direction that is at a non-zero angle to a central axis of the lumen.

4. The instrument of claim 1, wherein the electromagnetic sensor further comprises a second coil that is in the distal tip and defines a second area through which the lumen passes.

5. The instrument of claim 4, wherein the first area has a first normal direction, and the second area has a second normal direction that is at a non-zero angle to the first normal direction.

6. The instrument of claim 5, wherein the first normal direction is perpendicular to the second normal direction.

7. The instrument of claim 1, wherein the electromagnetic sensor further comprises a second coil that is in the distal tip and defines a second area through which a first radial axis of the instrument extends.

8. The instrument of claim 7, wherein the second area has a normal direction that is perpendicular to an instrument axis that extends along the lumen.

9. The instrument of claim 7, wherein the electromagnetic sensor further comprises a third coil that is in the distal tip and defines a third area through which a second radial axis of the instrument extends.

10. The instrument of claim 9, wherein the second radial axis is perpendicular to the first radial axis.

11. The instrument of claim 1, wherein the distal tip comprises a flexible material that defines a shape of the distal tip and encases the first coil.

12. A medical instrument comprising:
a main tube having a distal tip;
an electromagnetic sensor including a first coil that is in the distal tip and defines a first area, wherein a first radial axis that extends from a central axis of the main tube passes through the first area.

13. The instrument of claim 12, wherein the electromagnetic sensor further comprises a second coil that is in the distal tip and defines a second area, wherein a second radial axis that extends from the central axis of the main tube passes through the second area.

14. The instrument of claim 13, wherein the second radial axis is perpendicular to the first radial axis.

15. The instrument of claim 11, wherein the distal tip comprises a flexible material that defines a shape of the distal tip and encases the first coil.

16. The instrument of claim 11, wherein the main tube comprises a lumen that extends through the distal tip.

17. A medical instrument comprising:
a main tube having a distal tip; and
an electromagnetic sensor including:
a first coil that is in the distal tip and defines a first area having a first normal direction; and
a second coil that is in the distal tip and defines a second area having a second normal direction that is perpendicular to the first normal direction.

18. The instrument of claim 17, wherein the first normal direction is along a central axis of the main tube.

19. The instrument of claim 18, wherein the second normal direction is along a radial axis that extends from the central axis.

20. The instrument of claim 17, wherein a tool channel lumen of the instrument extends through the first area.

21. The instrument of claim 20, wherein the tool channel lumen extends through the second area.

22. The instrument of claim 17, wherein the first normal direction is along a radial axis that extends from a central axis of the main tube.

23. A method comprising:
generating a variable magnetic field with a known orientation with respect to anatomy of a patient;
placing an instrument in the patient within the magnetic field, wherein the instrument defines an interior lumen, and wherein an electromagnetic sensor in a distal tip of the instrument comprises a first coil that winds around the interior lumen; and
using an electrical signal induced in the first coil to measure and compute a position or orientation of the distal tip.

24. The method of claim 23, wherein the instrument further comprises a second coil in the distal tip,
wherein the second coil defines an area through which passes a radial axis that extends from a central axis of the catheter, and
wherein the method further comprises using an electrical signal induced in the second coil to measure and compute a position or orientation of the distal tip.
